(19) United States
(12) Patent Application Publication
(10) Pub. No.: US 2009/0193529 A1
(43) Pub. Date: Jul. 30, 2009

(54) Method for Generating Antibodies

(76) Inventors: Jill Giles-Komar, Downingtown, PA (US); Roberta A. Lamb, Wynnewood, PA (US); M. Lamine Mbow, King of Prussia, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/369,958

(22) Filed: Feb. 12, 2009

Related U.S. Application Data
(62) Division of application No. 11/414,106, filed on Apr. 28, 2006.
Application No. 11/414,106 is a division of application No. 10/644,308, filed on Aug. 20, 2003, now abandoned.

(51) Int. Cl. C12P 21/00

(52) U.S. Cl. 800/4

(57) ABSTRACT

Methods for generating antibodies in rodents are disclosed. The antibodies are useful as therapeutic agents, diagnostic agents or research reagents.

### CROSS-REFERENCE TO RELATED APPLICATION

This application is a divisional of U.S. Ser. No. 10/644,308 filed Aug. 20, 2003.

### FIELD OF THE INVENTION

This invention relates to the generation of antibodies in a rodent.

### BACKGROUND OF THE INVENTION

The use of monoclonal antibodies (mAbs) as therapeutic reagents has become an effective approach for the treatment of various diseases. In addition, mAbs can represent a powerful tool to gain a better understanding of the immunopathogenesis of various diseases.

A standard method for the generation of mAbs consists of fusing myeloma cells with lymph node cells or splenocytes harvested from immunized BALB/c mice (Köhler and Milstein, Nature 256, 495-497 (1975); Köhler and Milstein, Eur. J. Immunol. 6, 511-519 (1976)). BALB/c mice represent the host of choice for raising mAbs since they are readily available and, when sensitized with foreign T-dependent antigens, the immune response in these mice is characterized by a polarization of T-cell derived cytokine production toward a Th2-like phenotype (reviewed in Reiner and Locksley, Ann. Rev. Immunol. 13, 151-177 (1995)). This Th2-like response is accompanied by the generation of high levels of antigen-specific IgG1 antibodies (Finkelman et al., Ann. Rev. Immunol. 8, 303-333 (1990)), which correlates with an increase in the frequency of antigen-specific B-cell clones and an increase in the number of hybrids following B-cell fusion. Nevertheless, some antigens produce only low or undetectable antibody titers in BALB/c mice making it difficult or impossible to generate hybrids following B-cell fusion.

Advances in transgenic and gene knockout mouse models have provided new ways to make mAbs that are less immunogenic and to study the biology of immune-mediated responses. For example, mice transgenic for human immunoglobulin heavy and light chain genes can be used to generate fully human mAbs for therapeutic use (Lonberg et al. Nature 368, 856-859 (1994); Green, J. Immunol. Meth. 231, 11-23 (1999)). Gene knockout mice can be used to efficiently generate autologous mAbs against mouse proteins by circumventing immune tolerance of the targeted protein.

Transgenic and knockout mice are not from a BALB/c background. These mice are generally derived from a C57BL/6 (B6) background (The Jackson Laboratory catalog, 2001). However, the B6 genetic background does not represent the optimal immune environment for the generation of mAbs. This is due to the fact that the immune response in antigen-primed B6 mice is Th1-biased, which is characterized by a strong cellular response and a weak humoral response. Therefore, the generation of mAbs using B-cells harvested from B6 mice can be hindered by the low frequency of antigen-specific B-cell clones. While the use of adjuvants such as complete Freund's adjuvant or alum can boost the humoral response against foreign antigens, this procedure can denature some protein antigens. This can have a detrimental effect on the processing and presentation of key immunogenic epitopes for the generation of neutralizing antibodies. Further, the generation of mAbs against some antigens may prove difficult due to toxicity issues following repeated injections.

Thus, a need exists for methods that can rapidly generate high titers of antigen-specific antibodies in rodents such as BALB/c mice or in rodents not having a BALB/c background, such as B6 mice.

### BRIEF DESCRIPTION OF THE DRAWINGS

FIG. 1 shows a C57BL/6 mouse immunization schedule.

FIG. 2 shows anti-ovalbumin antibody production 9 days post-immunization.

FIG. 3 shows anti-ovalbumin antibody production 15 days post-immunization.

FIG. 4 shows a BALB/c mouse immunization schedule.

### SUMMARY OF THE INVENTION

One aspect of the invention is a method for generating monoclonal antibodies in a rodent comprising the steps of administering a dendritic cell expansion agent to the rodent; administering a dendritic cell maturation agent to the rodent; immunizing the rodent with an antigen; and isolating antigen-specific antibodies.

Another aspect of the invention is a method for generating monoclonal antibodies in a rodent comprising the steps of administering a dendritic cell maturation agent to the rodent; immunizing the rodent with an antigen; and isolating antigen-specific antibodies.

Another aspect of the invention is a method for generating antibodies in a C57BL/6 mouse comprising the steps of administering Flt3-L to the mouse; administering a combination of IFN-α and IFN-β to the mouse; immunizing the mouse with an antigen; and isolating antigen-specific antibodies.

Another aspect of the invention is a method for generating antibodies in a C57BL/6 mouse comprising the steps of administering Flt3-L to the mouse; administering a combination of IFN-α and IFN-β to the mouse; immunizing the mouse with an antigen; administering a CD40 agonist; and isolating antigen-specific antibodies.

A further aspect of the invention is a method for generating antibodies in a BALB/c mouse comprising the steps of administering a combination of IFN-α and IFN-β to the mouse; immunizing the mouse with an antigen; administering a CD40 agonist; and isolating antigen-specific antibodies.

### DETAILED DESCRIPTION OF THE INVENTION

All publications, including but not limited to patents and patent applications, cited in this specification are herein incorporated by reference as though fully set forth.

The term “antibodies” as used herein and in the claims means polyclonal, monoclonal or anti-idiotypic antibodies.

The term “antigen” as used herein and in the claims means any molecule that has the ability to generate antibodies either directly or indirectly. Included within the definition of “antigen” is a protein-encoding nucleic acid.

The term “dendritic cell expansion agent” as used herein and in the claims means any agent that causes the proliferation of immature dendritic cells.

The term “dendritic cell maturation agent” as used herein and in the claims means any agent that causes the conversion of immature dendritic cells to cells that can process antigens and display antigen peptide fragments on the cell surface together with molecules required for T-cell activation, known in the art as antigen-presenting cells (APC).

The term “in combination with” as used herein and in the claims means that the described agents can be administered to a rodent together in a mixture, concurrently as single agents or sequentially as single agents in any order.

The present invention provides methods for generating antibodies in rodents. In particular, the methods are useful for generating antibodies in rodents such as mice having a BALB/c background or not having a BALB/c background such as C57BL/6 (B6) mice.

In one embodiment of the present invention, expansion of dendritic cell numbers followed by administration of a dendritic cell maturation agent to a rodent that does not have a BALB/c background concurrent with or prior to immunization with foreign, T-dependent antigens enhances the humoral response and elicits a rapid and increased antibody response. This method of the invention is useful in the generation of antigen-specific IgG1 mAbs in these animals. The antibodies generated by the method of the invention are useful as therapeutic agents, diagnostic agents or research reagents.

In this embodiment of the invention, a dendritic cell expansion agent is administered to the rodent to achieve expansion of dendritic cell numbers. An expansion agent useful in the method of the invention is the tyrosine kinase receptor ligand Flt3 (Flt3L) (Lyman et al., Cell 75, 1157-1167 (1993)). Flt3L has been shown to increase dendritic cell numbers when injected into mice (Maraskovsky et al., J. Exp. Med. 184, 1953-1962 (1996)).

One of ordinary skill in the art could readily determine the amounts of Flt3L to administer. For example, about 8.8 μg to about 10 μg of Flt3L/day over a period of about 10 days to about 14 days can be used to induce dendritic cell maturation in mice. Flt3L can be adminstered singly or in combination with other dendritic cell expansion agents.

Further, in this embodiment of the invention, a dendritic cell maturation agent is administered to the rodent after administration of the expansion agent. Maturation agents useful in the method of the invention include any cytokines that will cause the conversion of dendritic cells to antigen-presenting cells and potentiate T-cell activation. These agents include type I interferons, tissue necrosis factor-α, interleukin-6, prostaglandin-E2, interleukin-1α, interleukin-1β, interleukin-18, interleukin-12, interleukin-4, interleukin-23, interferon-γ, granulocyte-macrophage colony-stimulating factor or a dendritic cell-associated maturation factor agonist singly or in combination with other dendritic cell maturation agents.

Dendritic cell-associated maturation factor agonists include, but are not limited to, any antibody, fragment or mimetic or small molecule agonist. An exemplary maturation factor agonist is an anti-CD40 antibody or antibody fragment such as a monoclonal anti-mouse CD40 antibody raised against a recombinant extracellular domain of mouse CD40.

Type I interferons include interferon-α (IFN-a), interferon-β (IFN-β), IFN-δ, IFN-α1, IFN-α2, IFN-α2a, IFN-α2b, IFN-α4, IFN-αII1, IFN-αCon1, IFN-αLE, IFN-αLy or IFN-β2. Type I interferon has been shown to induce antibody production (Le Bon et al., Immunity 14, 461-470 (2001).

One of ordinary skill in the art could readily determine the amounts of dendritic cell maturation agents to administer. For example, about 10⁵ U to about 2×10⁵ U each of IFN-α and IFN-β daily for about 3 days to about 5 days can be used to induce dendritic cell maturation.

Concurrent with or prior to administration of the dendritic cell maturation agent, the rodent is immunized with an antigen (protein or nucleic acid) by techniques well known to those skilled in the art. The antigen can be a protein or nucleic acid. In the case of protein antigens, adjuvant is not required.

Immunization of rodents with a nucleic acid antigen is a very effective method of generating high-titer antigen-specific IgG antibodies that recognize the native protein target. See Cohen et al., Faseb J. 12, 1611-1626 (1998), Robinson, Int. J. Mol. Med. 4, 549-555 (1999) and Donnelly et al., Dev. Biol. Stand. 95, 43-53 (1998). Exemplary plasmid vectors useful to contain the nucleic acid antigen with or without an adjuvant molecule contain a strong promoter, such as the HCMV immediate early enhancer/promoter or the MHC class I promoter, an intron to enhance processing of the transcript, such as the HCMV immediate early gene intron A, and a polyadenylation (polyA) signal, such as the late SV40 polyA signal. The plasmid can be multicistronic to enable expression of both the antigen and the adjuvant molecule, or multiple plasmids could be used that encode the antigen and adjuvant separately. An exemplary adjuvant is IL-4, others include IL-6, IFN-α, IFN-β and CD40.

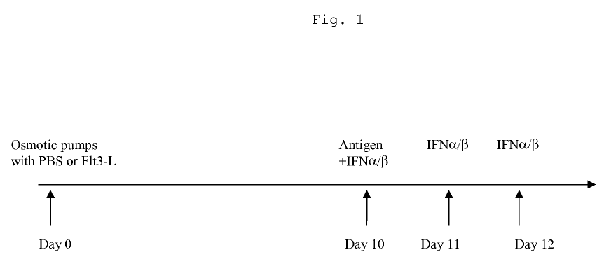

After immunization of the rodent, polyclonal antibodies or clonal populations of immortalized B cells are prepared by techniques known to the skilled artisan. Antigen-specific mAbs can be identified from clonal populations by screening for binding and/or biological activity toward the antigen of interest by using peptide display libraries or other techniques known to those skilled in the art. An exemplary immunization schedule for this embodiment of the invention is demonstrated in FIG. 1.

Optionally, in this embodiment of the invention, mice can be further treated post-immunization with an anti-CD40 agonist to enhance the immune response to antigens that produce low titers of antibodies. An exemplary anti-CD40 agonist useful in the method of the invention is an anti-mouse CD40 monoclonal antibody raised against the CD40 extracellular domain. One of ordinary skill in the art could readily determine the amounts of anti-CD40 antibody to administer. For example, about 50 μg to about 100 μg of the anti-CD40 mAb (clone 1C10) available from R&D Systems (Minneapolis, Minn.) under Catalog No. MAB440 administered at about day 14 post-immunization can be used to enhance the immune response in these mice.

In another embodiment of the invention, administration of a dendritic cell maturation agent to a rodent having a BALB/c background concurrent with or prior to immunization with foreign, T-dependent antigens enhances the humoral response and elicits a rapid and increased antibody response. This method of the invention is useful in the generation of antigen-specific IgG1 mAbs in these animals. The antibodies generated by the method of the invention are useful as therapeutic agents, diagnostic agents or research reagents.

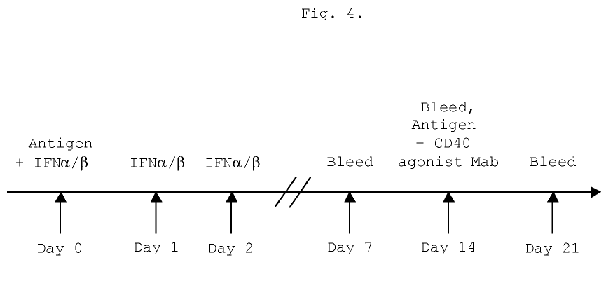

In this embodiment of the invention, the considerations for the dendritic cell maturation agent are identical to those discussed supra. Further, mice can optionally be further treated post-immunization with an anti-CD40 agonist to enhance the immune response to antigens that produce low titers of antibodies as discussed supra. An exemplary immunization schedule for this embodiment of the invention is shown in FIG. 4.

Given the rapidity and the amplitude of the immune response observed in treated mice, the methods of the invention can be used to immunize against a variety of immunogens including weak immunogens and potentially toxic antigens. In addition, the omission of adjuvant in the preparation of protein antigens should allow for processing and presentation of those conformational epitopes for targeting by neutralizing antibodies.

The present invention can also be used to boost the humoral response in immunodeficient mice from a B6 background reconstituted with human cells. For example, severe combined immunodeficient (SCID) mice and recombination activation gene deficient (RAG2−/−) mice can be used in the method of the invention. Human mAbs can be derived from these mice after reconstitution with human immune cells and immunization with antigen.

The present invention will now be described with reference to the following specific, non-limiting examples.

### Example 1

### Generation of Antigen-Specific mAbs in B6 Mice

Antibodies were generated in a series of various B6 mouse treatment groups against ovalbumin (OVA) as shown in Table 1. The immunization schedule is shown in FIG. 1. Carrier-free murine Flt3L (aa residues 1-188 described in Lyman et al., 1993, supra) and recombinant murine IFNα and IFNβ were purchased from R&D Systems (Minneapolis, Minn.). ALZET® osmotic pumps were purchased from Alza Corporation (Mountain View, Calif.). B6 mice (8 to 12 weeks old) were purchased from The Jackson Laboratory (Bar Harbor, Me.).

Osmotic pumps filled with Flt3L (100 μl per pump) were placed into the peritoneal cavity of mice. Control mice received pumps filled with PBS. Pumps delivered 8.8 μg of Flt3L/day/mouse over a period of 14 days. At day 10 following implantation of the pumps, some mice received one single subcutaneous injection of OVA in PBS (50 μg in the base of the tail). Depending on the treatment group (Table 1) some mice received daily injections of a mixture of IFN-α and IFN-β (IFN-α/β) starting the same day mice were immunized with the immunogen OVA. Mice received two more injections of IFN-α/β on days 1 and 2 post-OVA immunization. A total of 10⁵ U of IFN-A and 10⁵ U of IFN-β were injected into each mouse over a 3-day period.

TABLE 1
Treatment Groups

| | Group 1 | Group 2 | Group 3 |
|---|---|---|---|
| | (n = 3) | (n = 3) | (n = 3) |
| Pumps + PBS | Yes | No | No |
| Pumps + Flt3L | No | Yes | Yes |
| IFN-α/β | No | No | Yes |
| OVA | Yes | Yes | Yes |

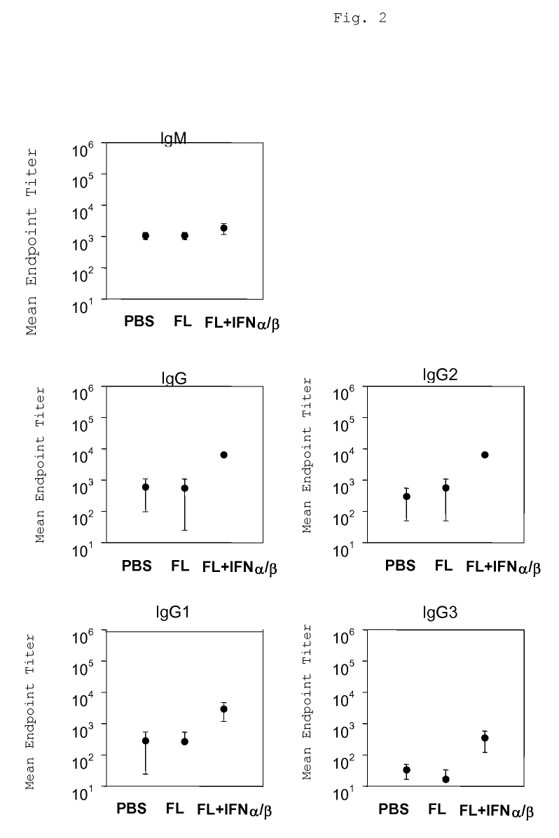

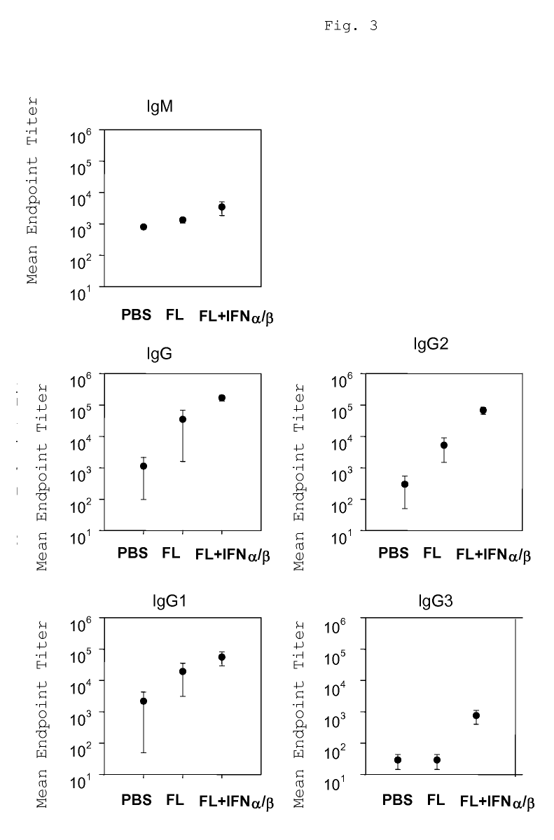

Levels of OVA-specific antibodies were determined by standard ELISA. The results in FIG. 2 demonstrate an increase in the levels of anti-OVA IgG antibodies at day 9 post-OVA immunization, with mice in the treatment Group 3 (Flt3L and IFN-α/β) showing the highest titers of OVA-specific IgG Abs. At day 15 post-OVA immunization (FIG. 3), anti-OVA IgG endpoint titers reached 2×10⁵ in all 3 mice in treatment Group 3. An increase in all IgG isotypes was observed in Group 3 mice.

### Example 2

### Generation of Antigen-Specific B Cells in B6 Mice

To assess the relative frequency of antigen-specific B cells, mice were given an intraperitoneal injection (1 mg/mouse) of bromodeoxyuridine (BrdU) one day prior to a soluble intravenous booster injection with OVA (15 μg/mouse). In addition, mice were fed with BrdU in their drinking water (0.5 mg/ml) starting one day prior to the soluble OVA booster injection. Splenocytes were obtained from mice three days after the soluble OVA booster injections and stained with FITC-labeled anti-BrdU and PE-labeled anti-B220. The relative frequency of B220+BrdU+ cells was determined by flow cytometric analysis.

As shown in Table 2, an increase in the frequency of B220+BrdU+ antigen-specific cells was observed in mice that were previously treated with Flt3L alone or Flt3L+IFN-α/β compared to mice that were given the pumps filled with PBS. The enriched populations of antigen-specific B cells observed in mice treated with Flt3L+IFN-α/β is expected to result in higher numbers of hybrids following B cell fusion and mAb production.

TABLE 2
Relative frequency of antigen-specific B220+ B cells

| Treatment Groups | % BrdU + B220+ |
|---|---|
| PBS | 10 |
| Flt3L | 19 |
| Flt3L + IFN-α/β | 16 |

### Example 3

### Up-Regulation of CD86 Expression on CD11c+ Dendritic Cells

To further define the immune mechanisms underlying the potent adjuvant effect of Flt3L+IFN-α/β, the frequency of mature CD11c+CD86+ dendritic cells was determined. Osmotic pumps were filled with either PBS or Flt3L and were implanted in the peritoneal cavity of separate groups of B6 mice (the various treatment groups are shown in Table 3). Osmotic pumps were set to deliver 8.8 μg Flt3L/mouse/day for 14 consecutive days. At day 10, all mice were injected subcutaneously at the base of the tail with OVA (15 μg/mouse). Mice that did receive OVA with IFN-α/β (10⁵ units each) at day 10 were also given two similar injections of IFN-α/β at day 11 and 12 (FIG. 1). Spleens were collected at day 13 for flow cytometric analysis of various dendritic cell populations. The relative percentage of the indicated dendritic cell populations is shown in Table 4. The results indicate that treatment with Flt3L+IFN-α/β resulted in an increase in the frequency of CD86+ (B7-2) CD11c+ splenic dendritic cells. These results suggest that the ability of Flt3L+IFN-α/β to increase levels of antigen-specific IgG antibodies may depend, at least in part, on the increase in the frequency of mature CD11c+CD86+dendritic cells.

TABLE 3
Treatment groups

| | PBS | Flt3L | Flt3L + IFN-α/β | IFN-α/β |
|---|---|---|---|---|
| | (n = 3) | (n = 3) | (n = 3) | (n = 3) |
| Pumps + PBS | Yes | No | No | Yes |
| Pumps + Flt3-L | No | Yes | Yes | No |
| IFN-α/β | No | No | Yes | Yes |
| OVA | Yes | Yes | Yes | Yes |

TABLE 4
Relative frequency of splenic dendritic cell populations

| | PBS | Flt3L | Flt3L + IFN-α/β | IFN-α/β |
|---|---|---|---|---|
| CD11c/CD40 | 4.3 | 7.5 | 9.1 | 4.2 |
| CD11c/CD8α+ | 1.5 | 8.7 | 9.6 | 3.4 |
| CD11cCD8α− | 2.8 | 5.7 | 7.8 | 6.5 |
| CD11c/CD80 | 2.8 | 6.7 | 7.2 | 4.5 |
| CD11c/CD86 | 3.8 | 8.3 | 12.3 | 7.2 |
| DEC205/CD40 | 7.5 | 7.7 | 5.5 | 4.4 |

### Example 4

### Enhancement of Antigen-Specific Titers by Anti-CD40 Treatment in BALB/c Mice

Antibodies were generated in two BALB/c mouse treatment groups against a humanized anti-CD3 monoclonal antibody (U.S. Pat. No. 6,491,916) as shown in Table 5. Anti-murine CD40 agonist monoclonal antibody (clone 1C10) was purchased from R&D Systems (Minneapolis, Minn.) under Catalog No. MAB440. All other reagents were sourced as previously described. BALB/c mice (8 to 12 weeks old) were purchased from The Jackson Laboratory (Bar Harbor, Me.).

The immunization schedule for the IFN-α/β+anti-CD40 treatment group is shown in FIG. 4. On day 0, mice were immunized subcutaneously (s.c.) with 25 μg humanized anti-CD3 mAb in the base of the tail and injected with a mixture of IFN-A and IFN-β (IFN-α/β). Mice received two more injections of IFN-α/β on days 1 and 2 post-immunization. A total of 10⁵ U of IFN-A and 10⁵ U of IFN-β were injected over the 3 day period. On day 14 post-immunization, mice were boosted with a s.c. injection of humanized anti-CD3 mAb and received a s.c. injection of anti-murine CD40 antibody (100 μg). All the mice were bled and titered on days 7, 14 and 21.

In the IFN-α/β+Flt3L treatment group, mice were immunized subcutaneously (s.c.) with 25 μg humanized anti-CD3 mAb in the base of the tail on day 0 and the remainder of the immunization schedule was similar to that described in Example 1.

As demonstrated in Table 5, at the end of the injection schedule the mice receiving humanized anti-CD3 mAb along with anti-CD40 treatment had IgG titers as high as 1:25,000 with a mean IgG titer of 1:6,200. In contrast, the highest IgG titer reached in the mice that were not treated with anti-CD40 was only 1:80 with a mean IgG titer of 1:26.

TABLE 5
Enhancement of IgG Titers

| Treatment Group | Highest IgG Titer | Mean IgG Titer |
|---|---|---|
| IFN-α/β + anti-CD40 | 1:25,000 | 1:6,200 |
| Flt3L + IFN-α/β | 1:80 | 1:26 |

The present invention now being fully described, it will be apparent to one of ordinary skill in the art that many changes and modifications can be made thereto without departing from the spirit or scope of the appended claims.

1. A method for generating antibodies in a C57BL/6 mouse comprising the steps of sequentially:
a) administering a Type 1 interferon dendritic cell maturation agent to the rodent;
b) immunizing the mouse with an antigen;
c) administering a CD40 agonist monoclonal antibody to the mouse; and
d) isolating antigen-specific antibodies.

2. (canceled)

3. The method of claim 2 wherein the type I interferon is interferon-α (IFN-α), interferon-β (IFN-β), IFN-δ, IFN-α1, IFN-α2, IFN-α2a, IFN-α2b, IFN-α4, IFN-αII1, IFN-αCon1, IFN-αLE, IFN-αLy or IFN-β2.

4. The method of claim 2 wherein the type I interferon is a combination of IFN-α and IFN-β.

5-14. (canceled)

15. The method of claim 4 wherein the IFN-α/β combination is administered in an amount of about 10⁵ U to about 2×10⁵ U each of IFN-α and IFN-β daily for about 3 days to about 5 days.

16. (canceled)

17. The method of claim 1 wherein the antibody is administered in an amount of about 50 μg to about 100 μg per dose.
